(19) United States
(12) Patent Application Publication
(10) Pub. No.: US 2006/0110730 A1
(43) Pub. Date: May 25, 2006

(54) Methods and compositions for regulation and manipulation of steroidogenesis

(76) Inventors: Himangshu S. Bose, Sunnyvale, CA (US); Vishwanath R. Lingappa, San Francisco, CA (US); Walter L. Miller, San Francisco, CA (US)

Correspondence Address: FOLEY & LARDNER LLP, 1530 PAGE MILL ROAD, PALO ALTO, CA, 94304, US

(21) Appl. No.: 10/510,593

(22) PCT Filed: May 20, 2003
(86) PCT No.: PCT/US03/15906

§ 371 Date: Sep. 19, 2005

Related U.S. Application Data
(60) Provisional application No. 60/382,359, filed on May 20, 2002.

(51) Int. Cl. C12Q 1/68; C07H 21/04; C12P 33/00; C12P 21/06; C07K 14/72

(52) U.S. Cl. 435/6; 435/69.1; 435/320.1; 435/325; 530/350; 536/23.5; 435/52

(57) ABSTRACT

The steroidogenic acute regulatory protein (StAR), a mitochondrial protein required for stress responses, reproduction, and sexual differentiation of male fetuses is shown to exert its activity transiently at the outer mitochondrial membrane (OMM) rather than in its final resting place in the matrix, and that its OMM residency time and activity are regulated by its speed of mitochondrial import. This may be the first example of a mitochondrial protein exerting its biological activity in a compartment other than that to which it is finally targeted. This unique system which permits steroidogenic cells to initiate and terminate massive levels of steroidogenesis within a few minutes, permitting the rapid regulation of serum steroid hormone concentrations therefore can be manipulated by altering the binding of the leader sequence for the StAR protein to its receptor on the OMM.

### GOVERNMENT SUPPORT

This invention was supported by NIH grants DK37922, DK02762 and NS37365. The US Government has rights in this invention.

### INTRODUCTION

1. Field of the Invention

The field is related to compositions and methods for identifying and using the site of regulation of transport of cholesterol into the mitochondria of a steroidogenic cell. The methods are exemplified by identification of a receptor on the outer mitochondrial membrane as the site of biological action of the StAR protein and the use of fusions of the StAR protein that alter its time on the outer mitochondrial membrane to alter the activity of the StAR protein thereby altering the rate of steroidogenesis.

2. Background

Hormonally induced, cAMP-mediated acute regulation of steroid hormone biosynthesis in steroidogenic cells is characterized by the mobilization of cholesterol from cellular stores to the outer membrane mitochondria, and the translocation of cholesterol to the inner membrane of the mitochondria where conversion of cholesterol to pregnenolone occurs. This process is regulated by the steroidogenic acute regulatory protein (StAR). StAR expression is restricted to organs that carry out mitochondrial sterol hydroxylation reactions that are under acute regulation by trophic hormones that act via the intermediacy of cAMP such as the adrenals and gonads which respond to their respective pituitary topic hormones, ACTH and LH with enhanced cholesterol side-chain cleavage and to the kidney which increases 1.α.hydroxylation of vitamin D in response to PTH. The production of mineralocorticoids, glucocorticoids, and sex hormones in steroidogenic tissues is dependent on the expression of StAR.

During import, mitochondrial proteins are typically processed by protein-import machinery consisting of Tom (translocase, outer membrane) proteins associated with the outer mitochondrial membrane (OMM), and Tim (translocase, inner membrane) proteins associated with the inner mitochondrial membrane (IMM) (Schatz, G. & Dobberstein, B., Science 271, 1519-1526 (1996); Neupert, W., Annu Rev Biochem, 863-917 (1997); Bauer, M. F. & Neupert, W., J Inher Metab Dis 24, 166-180 (2001)) and are directed to one of four submitochondrial compartments, OMM, IMM, intramembranous space (IMS), or matrix, where the protein then functions. Like most mitochondrial proteins, StAR is synthesized on cytoplasmic ribosomes and imported into the mitochondria where it is targeted to the matrix. However, the mechanism and site of action of StAR are controversial and it would be of interest to develop methods and compositions for determining its mechanism and site of action. Also of interest are methods of modulating steroidogenesis in vivo and methods of producing steroid hormones in vitro.

### SUMMARY OF THE INVENTION

Methods and compositions are provided for identifying the site of biological action of a mitochondrial protein that regulates steroidogenesis using a cell-free transcription/translation system and to compositions so identified and methods of using them. Also provided are methods and compositions for modulating steroidogenesis, increasing production of steroid hormones, and identifying specific agents that alter the rate of steroidogenesis. Methods for identifying the site of biological action of a mitochondrial protein that regulates steroidogenesis include the steps of expressing a mitochondrial protein of interest as a fusion protein with a means of immobilizing the protein of interest in one of the four mitchondrial compartments, and measuring the amount of steroid production associated with each immobilized protein of interest in a cell-free system. The receptor binding protein for a mitochondrial protein such as StAR can be used to screen for ligands that interact with the receptor, including agonists and antagonists that find use in regulating steroidogenesis and other activities associated with interaction between the mitochondrial protein and the receptor binding protein. Interaction of one or more subunits of the receptor binding protein and/or binding to the mitochondrial protein can be used as a marker of activation of the steroidogenesis pathway and blockade of this pathway can be used to block or enhance enzymes in the activated pathway, including steroidogenesis.

### BRIEF DESCRIPTION OF THE DRAWINGS

FIG. 1 shows that affixing StAR to the OMM increases steroidogenic activity. FIG. 1a, Steroidogenic activity (accumulated pregnenolone secreted after 48 h), of COS-1 cells co-transfected with the F2 fusion of the cholesterol side-chain cleavage system and with vectors expressing the indicated constructions. 22R-OH: steroidogenesis from control cells incubated with 22R-OH cholesterol; all other data show steroidogenesis from endogenous cellular cholesterol. FIG. 1b, Import of ³⁵S-StAR into MA-10 mitochondria. After 2 hours some of the 37 kDa cell-free translation product is incorporated into the membrane and cleaved to a 30 kDa protein, which remains membrane-associated. FIG. 1c, Import of StAR into MA-10 mitochondria. Extramitochondrial 37 kDa StAR is partially sensitive to proteinase K, but intramitochondrial 30 kDa StAR accumulates with time and remains protease-protected without Triton X-100. FIG. 1d, Import of Tom/StAR. 43 kDa Tom/StAR associates with mitochondria but is not imported; carbonate and digitonin extractions show association with OMM (cyto, cytoplasm; mito, mitochondria; sup, supernatant. FIG. 1e, Digitonin selectively solubilizes the IMM. MA-10 mitochondria were prepared, washed and serially extracted with carbonate and digitonin; the indicated fractions were separated by gel electrophoresis and immunoblotted with a mixture of antisera to human P450 scc and to human Tom20.

FIG. 2 shows that StAR is inactive in the IMS. FIG. 2a, Import of Tim9/StAR. 41 kDa Tim9/StAR is associated with mitochondria and with mitoplasts, but extraction of mitoplasts with NA₂CO₃ or their incubation with protease shows that Tim9/StAR resides on the IMS side of the IMM. FIG. 2b, The presence of Tim9/StAR in the IMS does not disrupt the OMM or the import and cleavage of full-length StAR. FIG. 2c, Reconstitution of mitochondrial activity from OMM and mitoplast fractions. Fractions containing StAR constructs are indicated by dashes, mixing of separate components is indicated by plus signs; the system is active only with Tom/StAR affixed to the OMM or with exogneously added StAR. FIG. 2d, StAR expressed in the transcription/ translation system is imported into mitochondria and cleaved to a 30 kDa form in mitoplasts, but when StAR is immunoprecipitated (IP) from the transcription/translation system it cannot be incorporated into mitochondria. FIG. 2e, Tim9/StAR immunoprecipitated from the transcription/translation system is nonetheless active when added to MA-10 mitochondria in vitro. Controls include immunoprecipitation of the transcription/translation system expressing full-length StAR, N-62 StAR, or expressing no StAR, the StAR mutant R182L (inactive in vivo (Bose, H. S., et al., N Engl J Med 335, 1870-1878 (1996)) and in vitro (Bose, H. S., et al., Biochemistry 39, 11722-11731 (2000)), buffer alone, use of heat treated mitochondria and use of cell-free transcription/translation system without added plasmid (mock). FIG. 2f, Steroidogenesis of MA-10 mitochondria incubated for various times with a non-radioactive cell-free transcription/translation system without added vector (control) and with equal amounts of StAR, N-62 StAR, Tom/StAR and Tim9/StAR, as assayed by incorporation of ³⁵S-methionine in a parallel experiment.

FIG. 3 shows the association of StAR activity with mitochondrial import. FIG. 3a, Steroidogenesis of COS-1 cells co-transfected with F2 and the indicated vectors. Fusion of 1-193 StAR to 63-285 StAR (StAR/StAR), yields maximal activity (equal to 22R-OH-cholesterol); Scc/N-30 StAR, Scc/N-62 StAR, and del StAR yieldreduced activity. StAR/StAR is StAR fused to 63-285 StAR with a Bam/EK linker. FIG. 3b, Time-course of import and cleavage (in minutes or hours). FIG. 3c, Effect of incubation temperature on import kinetics (in minutes) of full-length StAR into MA-10 mitochondria. FIG. 3d, Kinetic analysis of data in FIG. 3b; data are mean±s.d. from three independent experiments. FIG. 3e, Scc/N-30 StAR and Scc/N-62 StAR are imported and cleaved to intramitochondrial 30 kDa protein that is protected from protease digestion in the absence of detergent.

FIG. 4 shows that full-length and N-62 StAR are equally active. FIG. 4a, Equimolar amounts of empty vector (triangles), vector expressing full-length StAR (closed circles) and N-62 StAR (open circles) were transfected into COS-1 cells and the accumulated pregnenolone in the culture medium was measured at the times shown. FIG. 4a and FIG. 4b, Immunoblot of equimolar amounts of full-length, N-62, Scc/N-62 and Scc/N-30 StAR. FIG. 4c, Activity of MA-10 cell mitochondria incubated for 1h with equal masses of each protein prepared by cell-free transcription/translation.

FIG. 5 shows evidence for a StAR signal sequence-specific receptor protein on the OMM. ³⁵-labeled full-length StAR (WT) and SCC/N-62 StAR were incubated with a homobifunctional crosslinker, BS³, in the presence or absence of mitochondria at 26° and 4° C. A new protein crosslinked product of approximate 80 kDa size is present only with the full-length StAR at both 26° and 4° C., but not in the absence of mitochondria (extreme left lane), or if the StAR signal sequence and amino acids 1-62 (containing the putative pause region) is replaced by that of side chain cleavage enzyme (SCC) (see right hand panel).

FIG. 6 shows StAR signal sequence-specific inhibition of protein import into mitochondria. Import of StAR, but not of a chimera containing SCC leader on mature StAR sequence, was blocked by a VDAC-specific inhibitor indicating that VDAC represents an alternate mitochondrial import channel utilized by StAR and possibly other substrates. 50 μl of StAR or SCC-StAR translation product was mixed with 250 μg of mitochondria prepared by resuspending 10,000× g pellet from 10 million cell homogenate, import was carried out for 1 hr in the absence or presence of VDAC inhibitor (Koenig's polyanion) at 20 nM to 0.002 nM concentration points. After 1 hr, the reaction was placed on ice and aliquots analyzed for import by signal cleavage and proteolysis. The 50% inhibition point for StAR import is obtained with less than 4 pM Koenig's polyanion as compared to about 4 nM for SCC/N-62 StAR.

FIG. 7 shows that StAR and SCC-StAR display different protein-protein interactions on glycerol density gradients. Translation and import StAR and SCC-StAR translation products into mitochondria were carried out as described in Methods and a 20 μl aliquot was taken, solubilized in 1% lauryl maltoside and applied to 10-30% glycerol gradients containing 750 mM amino caproic acid pH 7.4 detergent and centrifuged in a TLS55 rotor for 2 or 6 hours, with 125 μl fractions taken and analyzed by SDS PAGE as shown.

FIG. 8. 100 μl Translation was solubilized with 1% maltoside and analyzed by native blue gel electrophoresis (reference). Bands observed to be coassociated with StAR but not SCC StAR were excised and analyzed by Matrix assisted laser desorption ionization/Time of flight (MALDI/TOF). The following products were identified with StAR but not SCC StAR: VDAC1 and VDAC3, adenine nucleotide translocator, aldehyde dehydrogenase, ADP, ATP carrier protein and glucose regulatory protein (GRP-78).

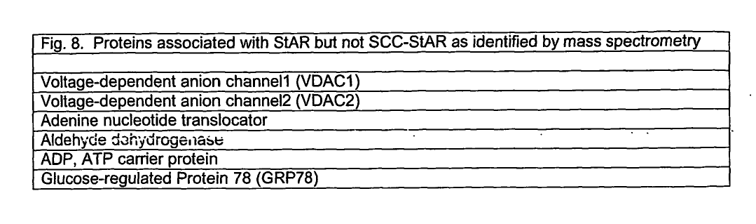

### DESCRIPTION OF THE SPECIFIC EMBODIMENTS

Methods and compositions for elucidating the site of biological activity in an organelle, such as the mitochondria, of a protein of interest using a cell free transcription-translation system to which isolated organelle has been added are described. Proteins of particular interest include those that catalyze a rate limiting step in a biological pathway, either of synthesis or degradation, such as a steroidogenesis pathway in steroidogenic cells in for example the ovary, testes and kidney, and an isolated mitochondrial StAR receptor binding protein comprising as a first subunit a voltage dependent anion channel (VDAC), particularly VDAC1 or VDAC3 obtainable from a steroidogenic cell, preferably a primate cell and more preferably a human cell. The StAR receptor binding protein can include one or more additional subunits, including one or more protein of an adenine nucleotide translocator, an aldehyde dehydrogenase, an ATP carrier protein and a glucose regulated protein 78. Also of interest is an isolated complex comprising a StAR leader sequence, or a fragment thereof that binds to VDAC and/or one or more subunits in the receptor binding protein and modulates steroidogenic activity. The one or more subunits preferably includes a VDAC. Also of interest are non-peptide ligands that bind to the StAR receptor binding protein and modulate steroidogeneis and/or other activities in the steroidogenesis pathway. The ligands can be agonists or antagonists of the receptor binding protein.

The cell-free methodology offers several advantages over existing systems as a means of localizing the site of action of a particular protein, particularly when that site is other than the site to which the protein is ultimately targeted. The process can be used to detect transmediates currently not detectable in any other way and to purify and/or isolate them. By “transmediates” is intended transient intermediates in a biological activity cascade. By identifying previously unknown intermediate complexes in a particular biological pathway, the specific binding pairs, such as receptors and their ligands, that form the complex can be evaluated for example as a means of altering the activity of the pathway, as putative drug targets for treating disease(s) associated with the biological pathway and as a potential source of symptoms associated with disease.

For the production and isolation of a significant quantity of functional proteins of interest host cells are transformed with recombinant vectors for the production of these proteins. The host cells may be genetically engineered cells from which naturally occurring genes for the proteins of interest have been substantially deleted. Host cells for the production of the functional proteins of interest effective in cell-free systems can be derived from any cells with the capability of harboring a recombinant protein of interest. However, preferred host cells are those that naturally produce the proteins of interest. Thus if the proteins of interest are enzymes in a steroidogenesis pathway that are targeted to the mitochondria of steroidogenic cells, such as leydig cells of the testes, such cells would be preferred host cells. Examples include cultured leydig cells such as the mouse MA-10 cell line. The host cells can be transformed with one or more vectors, collectively encoding one or more proteins of a set of functional proteins sufficient to effect production of an end product of the biological pathway to which the protein of interest belongs. The vector(s) can include native or hybrid combinations of pathway components (such as means of immobilizing a protein of interest in a particular cellular or organelle compartment of interest), and/or mutants thereof.

The recombinant vectors containing nucleic acid encoding the protein(s) of interest can be conveniently generated using techniques known in the art. For example, the gene encoding either the StAR protein or the receptor for the StAR leader sequence can be obtained from any cell that expresses the same, using recombinant methods, such as by screening cDNA or genomic libraries, derived from cells expressing the gene, or by deriving the gene from a vector known to include the same. The gene can then be isolated and combined with other desired nucleic acid sequences, using standard techniques. If the gene in question is already present in a suitable expression vector, it can be combined in situ, with, e.g., other nucleic acid sequences, as desired. The gene also can be produced synthetically, rather than cloned. The nucleotide sequence can be designed with the appropriate codons for the particular amino acid sequence desired. In general, one will select preferred codons for the intended host in which the sequence will be expressed. The complete sequence can be assembled from overlapping oligonucleotides prepared by standard methods and assembled into a complete coding sequence.

Mutations can be made to the native nucleic acid sequences and such mutants used in place of the native sequence, for example to identify regions required for biological activity. Such mutations can be made to the native sequences using conventional techniques such as by preparing synthetic oligonucleotides including the mutations and inserting the mutated sequence into the gene using restriction endonuclease digestion. Alternatively, the mutations can be effected using a mismatched primer (generally 10-20 nucleotides in length) which hybridizes to the native nucleotide sequence (generally cDNA corresponding to the RNA sequence), at a temperature below the melting temperature of the mismatched duplex. The primer can be made specific by keeping primer length and base composition within relatively narrow limits and by keeping the mutant base centrally located. Primer extension is effected using DNA polymerase, the product cloned and clones containing the mutated DNA, derived by segregation of the primer extended strand, selected. Selection can be accomplished using the mutant primer as a hybridization probe. The technique is also applicable for generating multiple point mutations. PCR mutagenesis also finds use for effecting the desired mutations.

The gene sequences, native or mutant, can be inserted into one or more expression vectors, using methods known to those of skill in the art. Expression vectors will include control sequences operably linked to the desired coding sequence. Suitable expression systems for use with the present invention include systems which function in eukaryotic host cells. Selectable markers can also be included in the recombinant expression vectors. A variety of markers are known which are useful in selecting for transformed cell lines and generally comprise a gene whose expression confers a selectable phenotype on transformed cells when the cells are grown in an appropriate selective medium. Such markers include, for example, genes which confer antibiotic resistance or sensitivity to the plasmid. Alternatively, where the product produced by the biological pathway of which the protein of interest is a component can easily be detected, this characteristic can be used as a marker for selecting cells that have been successfully transformed.

Methods for introducing the recombinant vectors of the present invention into suitable hosts are known to those of skill in the art and typically include the use of CaCl₂ or other agents, such as divalent cations and DMSO. The cells modified to contain the expression systems for functional proteins of interest are then cultured under conditions suitable for expression of the proteins of interest. If the proteins for use in the cell-free system are to be prepared recombinantly as described above, the cells producing the relevant proteins are optionally harvested and disrupted if the desired proteins have been intracellularly produced. However, if the expression system secretes the protein into growth media, the protein can be purified directly from the media.

If the protein is not secreted, it can be isolated from cell lysates. This is generally accomplished by first preparing a crude extract which lacks cellular components and several extraneous proteins. The desired proteins can then be further purified, for example by column chromatography, HPLC, immunoadsorbent techniques or other conventional methods well known in the art. The selection of the appropriate growth conditions and recovery methods are within the skill of the art. For example, cells that express the protein of interest can be grown to produce a predetermined number of cells. The cells are then disrupted by sonication, freeze-thaw cycles or other like techniques by which the cell membrane is breached to form a crude cell-free preparation. The crude cell-free preparation can be used at this stage as a source of the protein of interest or may be further processed by centrifugation, filtration or the like, to form a cell supernatant. Optionally, nucleic acids may be removed from the cell supernatant by, for example, precipitation with polyethyleneimine, or other like agent which does not disturb the biologic activity of the protein of interest. The preparation can be used at this stage as or optionally, the protein of interest can be further purified by techniques known to those of skill in the art. Isolated native forms of the protein of interest may in some instances be used. The purified protein of interest then can be used to catalytically synthesize a desired end product in a cell-free system as exemplified below. The cell-free system includes purified protein of interest in an appropriate buffer, and the substrates required for the catalytic synthesis of a desired end product.

Proteins that are targeted to a particular cellular organelle are proteins which have a signal or leader sequence which generally is a member of a specific binding pair, the second member being a receptor on the membrane associated with the target cellular organelle. The signal sequences are usually N-terminal, but may be internal to the protein or C-terminal. The protein with leader sequence can be used for the production of antibodies, either antisera or preferably monoclonal antibodies. The antisera and antibodies are prepared in conventional ways by immunizing a host, usually a mouse in the case of monoclonal antibodies, with or without an adjuvant, followed by additional injections of the protein at biweekly or longer intervals and monitoring the level of antisera. For monoclonal antibodies, splenocytes may be isolated, immortalized and screened. Those hybridomas which produce antisera which can block binding of the protein to the cellular organelle membrane are expanded. The antibodies may then be used to isolate the receptor protein by for example affinity chromatography.

The binding affinity for the members of the specific binding pair can be determined in assays, either homogeneous or non-homogeneous. Numerous protocols are available for detecting binding between a ligand and a receptor involved in complex formation. The two proteins may be labeled with a fluorescer and an energy receptor, so that binding of the two proteins together would reduce the level of emission at the wavelength of the fluorescer and increase the emission at the level of the energy acceptor. Alternatively, one may bind one of the proteins to a surface and the level of binding of the other protein to the bound protein is determined by having the second protein labeled. Alternatively, one may bind each of the proteins to different particles, where one particle produces a compound, which activates the other particle. Where the labeled entity is small, such as an oligomer or small organic molecule, a fluorescer may be used as the label and fluorescence polarization employed for detection. Illustrative of assays are the assays described in U.S. Pat. Nos. 5,989,921; 4,806,488; 4,318,707; 4,255,329; 4,233,402; and 4,199,559.

The location of binding of signal sequences also may be determined by analysis of crosslinking patterns generated when truncated transcripts encoding those signal sequences at the 5′ end of the authentic coding region of interest are expressed by cell-free translation and subject to chemical crosslinking including but not limited to lysine and cystiene specific cleavable and uncleavable crosslinkers, with analysis of the crosslink patterns by immunoprecipitation and polyacrylamide gel electrophoresis in sodium dodecyl sulfate and subsequent autoradiography.

To study binding to a particular cellular organelle, and as appropriate compartments within the organelle, the organelle is isolated from appropriate cells and then constructs that can be immobilized in the particular organelle are imported into the organelle using a cell free system. As an example, the conditions for cell-free translation for mitochondrial import of StAR are those reported previously (see Perara and Lingappa (1985) J. Cell Biol. 101:2292-2301) using a rabbit reticulocyte extract system. This system can be readily adapted to other translation systems such as the wheat germ translation system, or Xenopus oocyte translation system. The ability to mix and match components from multiple such fractionated systems allows tissue specific events involved in the biogenesis of the protein of interest to be studied. A number of cell-free systems are available commercially. For a review, see for example Wilkinson The Scientist (1999) 13: 15 and Smutzer The Scientist (2001) 15: 22. Also see www.ambion.com, www.novagen.com, and www.biochem.roche.com for kits for linked in vitro transcription/translation systems. Other references include U.S. Pat. Nos. 5,998,163; 5,998,136 and 5,989,833. The ancillary proteins associated with the ER and translocation of the translocation product may be removed from the lysate by methods described in the literature. (Gorlich, et al., (1992) Nature 357, 47-52; Gorlich and Rapoport, (1993) Cell 75, 615-630; and Hanein, et al., (1996) Cell 87, 721-732).

Non-peptide small molecules that can bind to the StAR leader sequence receptor binding protein or that may block binding of the leader sequence to the receptor binding protein may be screened by employing matrices to which the isolated receptor binding protein or leader sequence are bound and/or mutants of either of these proteins that may be associated with disease. See, for example, U.S. Pat. Nos. 5,631,734; 5,856,102 and 5,919,523. These matrices are available commercially and can be prepared in relation to a particular binding pattern i.e. the proteins on the matrices can be addressable. The matrix and antibodies may be used in conjunction to confirm the other assay, isolate the conformer, used together in the same assay. The proteins may be labeled with a detectable label, e.g. fluorescer, luminescer, phosphorescer, enzyme, radioisotope, and the like. The oligopeptides can be used in competitive assays for identifying other oligopeptides which compete for the site or other compounds, particularly small organic compounds, natural or synthetic, of less than 5 kDal, usually less than about 2.5 kDal. These compounds may then be used in turn to identify other compounds having greater affinity for the receptor binding protein and/or the leader Techniques for obtaining the DNA for the receptor binding protein for the StAR leader sequence receptor binding protein on the outer mitochondrial membrane are well known to those of skill in the art. Briefly, the DNA for the gene is isolated knowing the amino acid sequence and using degenerate probes. The amino acid sequence of the receptor binding protein protein can be obtained following isolation of the receptor binding protein has described above using methods known to those of skill in the art. The DNA sequences, which bind to the probes, are isolated and sequenced to identify a sequence coding for the protein of interest. If one wishes to avoid the presence of introns, the mRNA may be isolated, reverse transcribed and amplified using PCR. Once the gene is produced it may be introduced into one of numerous commercially available vectors and cloned and/or an expression vector may be employed having a transcriptional regulatory region 5′ of the sense strand to provide for expression. By using mammalian cells, the receptor binding protein can be produced and isolated and assayed as described above. In this way, significant amounts of the receptor binding protein may be isolated.

The following examples are offered by way of illustration of the present invention, not limitation.

### EXAMPLES

### Methods Construction of Vectors

Tom20, Tim9 and Tim44 complementary DNAs were prepared from human adrenal RNA by PCR after reverse transcription of RNA (RT-PCR). N-62 StAR cDNA including an enterokinase linker fused to 63-285 StAR was derived from the bacterial expression vector (Bose, H. S., et al. Incorrect folding of steroidogenic acute regulatory protein (StAR) in congenital lipoid adrenal hyperplasia. Biochemistry 37, 9768-9775 (1998)). Tom/StAR is Tom20 fused to 63-285 through GSDDDDK, which contains BamHI and enterokinase sites (Bam/EK linker). StAR/Tom is 1-285 StAR fused to Tom20 through EcoRI/EK linker (GFDDDDK). Tim9/StAR contains the 25-amino-acid mitochondrial leader of cytochrome c oxidase subunit IV (Hurt, E. C., et al. The first twelve amino acids (less than half of the pre-sequence) of an imported mitochondrial protein can direct mouse cytosolic dihydrofolate reductase into the yeast mitochondrial matrix. EMBO J. 4, 2061-2068 (1985)) fused to Tim9 linked to N-30 StAR through the Bam/EK linker. Tim44/StAR is Tim44 fused to 63-285 StAR through the Bam/EK linker. StAR/StAR is StAR fused to 63-285 StAR with the Bam/EK linker. For Scc/N-30 and Scc/N-62 StAR, cDNA encoding amino acids 1-39 of human P450scc (Chung, B., Matteson, et al. Human cholesterol sidechain cleavage enzyme, P450 scc: cDNA cloning, assignment of the gene to chromosome 15, and expression in the placenta. Proc. Natl Acad. Sci. USA 83, 8962-8966 (1986)) was fused directly to N-30 StAR or N-62 StAR. Del StAR is StAR residues 1-30 fused directly to 63-285. Constructs were verified by sequencing and expressed in the BamHI/EcoRI site of pCMV-Flag (Stratagene).

### Cell Culture

COS-1 cells were grown, transfected and assayed for pregnenolone production as Described (Stocco, D. M. & Clark, B. J. Regulation of the acute production of steroids in steroidogenic cells. Endocr. Rev. 17, 221-244 (1996)), (Bose, H. S., et al. N-218 MLN64, a protein with StAR-like steroidogenic activity is folded and cleaved similarly to StAR. Biochemistry 39, 11722-11731 (2000)). Three independent transfections, each performed in triplicate, were done for each construct.

### Protein Import Assays

StAR constructs were cloned into the BglII/EcoRI site of pSP6, transcribed with SP6 RNA polymerase (Promega) and translated with [35S]methionine as described (Chuck, S. & Lingappa, V. Apolipoprotein B intermediates. Nature 356, 115-116 (1992)). Mitochondria, isolated from 5 £ 106 MA-10 or COS-1 cells by hypotonic shock and homogenization (Bose, H. S., et al. N-218 MLN64, a protein with StAR-like steroidogenic activity is folded and cleaved similarly to StAR. Biochemistry 39, 11722-11731 (2000)) were suspended in 100 ml of 125 mM sucrose, 1 mM ATP, 1 mM NADH, 50 mM KCl, 0.05 mM ADP, 2 mM DTT, 5 mM Na-succinate, 2 mM Mg(OAc)2, 2 mM KH2PO4 and 10 mM HEPES buffer at pH 7.4. Mitochondria (50 ml) and 1 ml of the transcription/translationmix were incubated at 268 C. for various times; import was terminated with 1 ml 1 mM mCCCP (Calbiochem). Some mitochondria were extracted with fresh 100 mM Na2CO3, pH 11.5, for 15 min at 48 C. and spun at 15,000 g for 15 min at 48 C. to pellet membranes. Some membranes were incubated with 0.1% digitonin (Sigma) for 15 min at 48 C. and spun at 144,000 g for 1 h at 48 C. to pelletOMMfragments. Mitoplast preparation: mitochondria were incubated with 1U trypsin for 15 min at 48 C., washed with 50 mM NaCl, 10 mM HEPES pH 6.5 containing 10U soybean trypsin inhibitor and osmotically shocked in 10 mM HEPES pH 7.5 for 30 min at 48 C. (ref. 30). Analysis was by SDS-polyacrylamide gel electrophoresis (SDS-PAGE) and phosphorimager quantification (Molecular Dynamics, Storm 860).

### Activities in Isolated Mitochondria

StAR constructs were bioassayed (Bose, H. S., et al. N-218 MLN64, a protein with StAR-like steroidogenic activity is folded and cleaved similarly to StAR. Biochemistry 39, 11722-11731 (2000)) with MA-10 mitochondria. Linked transcription/translation system was added directly to the mitochondria except when Tim9/StAR was immunoprecipitated with anti-StAR antiserum (Bose, H. S., et al. The active form of the steroidogenic acute regulatory protein, StAR, appears to be a molten globule. Proc. Natl Acad. Sci. USA 96, 7250-7255 (1999)), collected with protein A-sepharose CL-4B (Pharmacia), washed, dissociated from the sepharose-antibody complex at pH 3.0, neutralized and added directly to mitochondria. Immunoassays of pregnenolone were performed after incubation at 378 C. for 1 h; immunoblotting was performed as described (Bose, H. S., et al. N-218 MLN64, a protein with StAR-like steroidogenic activity is folded and cleaved similarly to StAR. Biochemistry 39, 11722-11731 (2000)).

### EXAMPLE 1

### Affixing StAR to the Outer Mitochondrial Membrane Increases Steroidogenic Activity

To determine the location of biological activity of the mitochondrial protein StAR, wild-type and StAR proteins with an N-terminal deletion and StAR proteins fused to a protein that would function to immobilize the StAR protein in one of the four mitochondrial compartments were evaluated for their ability to catalyze steroidogenesis. Nonsteroidogenic COS-I monkey kidney cells were grown, transfected and assayed for pregnenolone production as described (Stocco, D. M. and Clark, B. J., Endocr Rev 17, 221-244 (1996); Bose, H. S., et al., Biochemistry 39, 11722-11731 (2000)). Three independent transfections, each performed in triplicate, were done for each construct. The cells were transfected with a vector expressing full length StAR or a vector expressing N-62 StAR, Tom-StAR, StAR-Tom, Tim44-StAR, or Tim9-StAR. Tom20, Tim9 and Tim44 cDNAs were prepared from human adrenal RNA by RT-PCR. N-62 StAR cDNA including an enterokinase linker fused to 63-285 StAR was derived from a bacterial expression vector (see Artemenko, et al., J. Biol. Chem. 276: 46583-46596 (2001). Tom/StAR is Tom20 fused to 63-285 through GSDDDDK, which contains Bam HI and enterokinase sites (Bam/EK linker). StAR/Tom is 1-285 StAR fused to Tom20 through Eco RI/EK linker (GFDDDDK). Tim9/StAR contains the 25 amino acid mitochondrial leader of cytochrome c oxidase subunit IV (Bose et al, Biochemistry, 37: 9768-9775 (1998)) fused to Tim9 linked to N-30 StAR through the Bam/EK linker. Tim44/StAR is Tim44 fused to 63-285 StAR through the Bam/EK linker. Tom20 has 50 amino-terminal residues embedded in the OMM and 95 residues in the cytoplasm (Abe, Y., et al., Cell 100, 551-560 (2000)). Expression of Tom20 with N-62 StAR fused to its carboxy-terminus (Tom/StAR) affixed StAR to the cytoplasmic side of the OMM. Expression of Tim9, which is normally confined to the IMS (Bauer et al, J. Inher. Metab. Dis. 24: 166-180 (2001)), fused to N-30 StAR (Tim9/StAR) localized StAR to the IMS. Expression of Tim44, which lies on the matrix side of the IMM, fused to N-62 StAR (Tim44/StAR), targeted StAR to the matrix. The cells were co-transfected with a pECE vector expressing a fusion protein termed F2, consisting of the human cholesterol side-chain cleavage system: HN-P450scc-Adrenodoxin Reductase-Adrenodoxin-COOH (J. A. Harikrishna et al., DNA Cell Biol. 12, 371 (1993)). The substrate was endogenous cellular cholesterol or added 22R-hydroxycholesterol (22R-OH.). After 48 h of incubation, the medium was collected and assayed for pregnenolone.

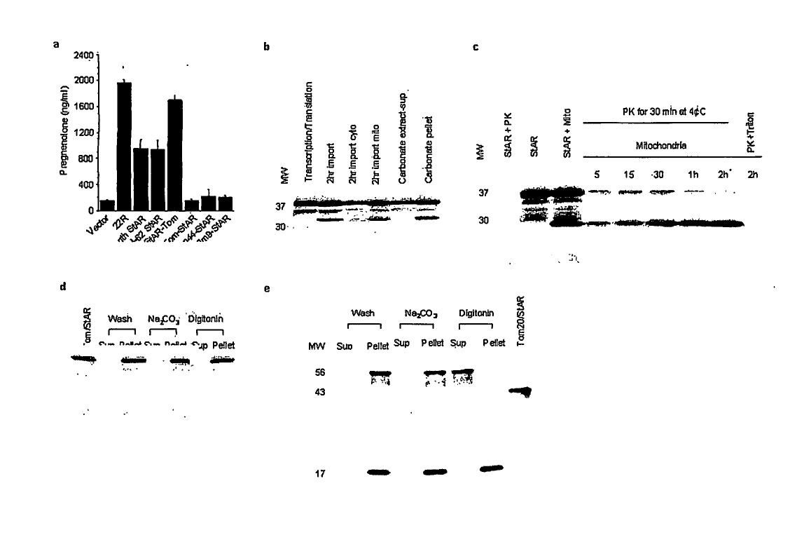

Western blots with antiserum to human StAR indicated that each StAR construct was expressed at equivalent levels (not shown). Expression of F2 alone showed low levels of StARindependent steroidogenesis. Incubation with 22R-OH-cholesterol, which bypasses the action of StAR, showed maximal steroidogenic capacity ˜20-fold greater than the StAR-independent level (FIG. 1a). Co-expression of F2 and full-length (1-285) StAR or N-62 StAR showed a 6-fold increase in steroidogenesis over the StAR-independent level seen with F2 alone or about half the maximal capacity of the cells seen with 22R-OHcholesterol. Co-expression of Tom/StAR and F2 achieved maximal steroidogenesis equal to that seen with exogenously added 22R-OH-cholesterol; by contrast StAR/Tom, Tim44/StAR, and Tim9/StAR did not increase steroidogenesis. Thus localizing StAR to the OMM substantially increased activity, but StAR localized to the IMS or matrix was inactive. The results are shown in FIG. 1a.

StAR constructs were bioassayed (Bose, H. S., et al., Biochemistry 39, 11722-11731 (2000)) with MA-10 mitochondria. Linked transcription/translation system was added directly to the mitochondria except when Tim9/StAR was immunoprecipitated with anti-StAR antiserum (Bose, H. S., et al., Proc Natl Acad Sci USA 96, 7250-7255 (1999)), harvested with protein A-sepharose CL-4B (Pharmacia), washed, dissociated from the sepharose-antibody complex at pH 3.0, neutralized and added directly to mitochondria. Immunoassays of pregnenolone were performed after incubation at 37° for 1 h; immunoblotting was performed as described (Bose, H. S., et al., Biochemistry 39, 11722-11731 (2000)).

StAR constructs were cloned into the Bgl II/Eco RI site of pSP6, transcribed with SP6 RNA polymerase (Promega) and translated with ³⁵S methionine as described (Chuck, S. and Lingappa, V., Nature 356, 115-6 (1992)). Mitochondria, isolated from 5×10⁶ MA-10 or COS-1 cells by hypotonic shock and homogenization (Bose, H. S., et al., Biochemistry 39, 11722-11731 (2000)) were suspended in 100 □l of 125 mM sucrose, 1 mM ATP, 1 mM NADH, 50 mM KCl, 0.05 mM ADP, 2 mM DTT, 5 mM Na-succinate, 2 mM Mg(OAc)₂, 2 mM KH2PO₄, pH 7.4. Mitochondria (50 μl) and 1 μl of the transcription/translation mix were incubated at 26° C. for various times; import was terminated with 1 μl 1 mM mCCCP (Calbiochem). Some mitochondria were extracted with fresh 100 mM NA₂CO₃, pH 11.5, for 15 min at 4° C. and spun at 15,000×g for 15 min at 4° C. to pellet membranes. Some membranes were incubated with 0.1% digitonin (Sigma) for 15 min at 4° and spun at 144,000× g for 1h at 4° C. to pellet OMM fragments.

Mitochondrial import assays (Rapoport, D. and Neupert, W., J Cell Biol 146, 321-331 (1999)) show that Tom/StAR is associated with the OMM. Full-length ³⁵S-StAR prepared in vitro by a linked transcription/translation system was imported into mouse Leydig MA-10 cell mitochondria and cleaved to a 30 kDa form associated with the mitochondrial pellet. Extraction with Na2CO3, which disrupts protein interactions but not lipid-protein interactions (Li, J. M. and Shore, G. C., Science 256, 1815-1817 (1992); Meisinger, C., et al., Mol Cell Biol 21, 2337-2348 (2001)), showed that the imported 30 kDa StAR form and some of 37 kDa protein were associated with lipid membranes (FIG. 1b). Extramitochondrial 37 kDa is only partially sensitive to proteinase K, suggesting that it is tightly associated with the OMM; intramitocondrial 30 kDa StAR is wholly insensitive to proteinase K, and membrane solubilization with Triton X-100 renders both forms protease-sensitive (FIG. 1c). ³⁵S Tom/StAR was also associated with mitochondria, as extraction with Na2CO3 did not dissociate ³⁵S Tom/StAR from OMM (FIG. 1d). Extraction of the post-carbonate pellet with digitonin dissolves the IMM but not the OMM (Meisinger, C., et al., Mol Cell Biol 21, 2337-2348 (2001); Regan C I, et al., In Mitochondria: A Practical Approach (eds. Darly-Usmar, V. M., Rickwood, D. & Wilson, M. T.) 79-112 (IRL Press, Washington D.C, 1987)) (FIG. 1e), but digitonin did not solubilize ³⁵S Tom/StAR (FIG. 1d). Thus Tom/StAR is incorporated into the outer but not the inner mitochondrial membrane where it exhibits substantially increased StAR activity, and this incorporation requires interaction with membrane lipids.

### EXAMPLE 2

### StAR is Inactive in the Intramembraneous Space

Mitoplasts were prepared as follows. Mitochondria were incubated with 1U trypsin for 15 min at 4° C., washed with 50 mM NaCl, 10 mM HEPES pH 6.5 containing 10U soybean trypsin inhibitor and osmotically shocked in 10 mM HEPES pH 7.5 for 30 min at 4° C. (Luciano, P., et al., EMBO J 20, 4099-4016 (2001)). Analysis was by SDS/PAGE and phosphorimager quantitation (Molecular Dynamics, Storm 860).

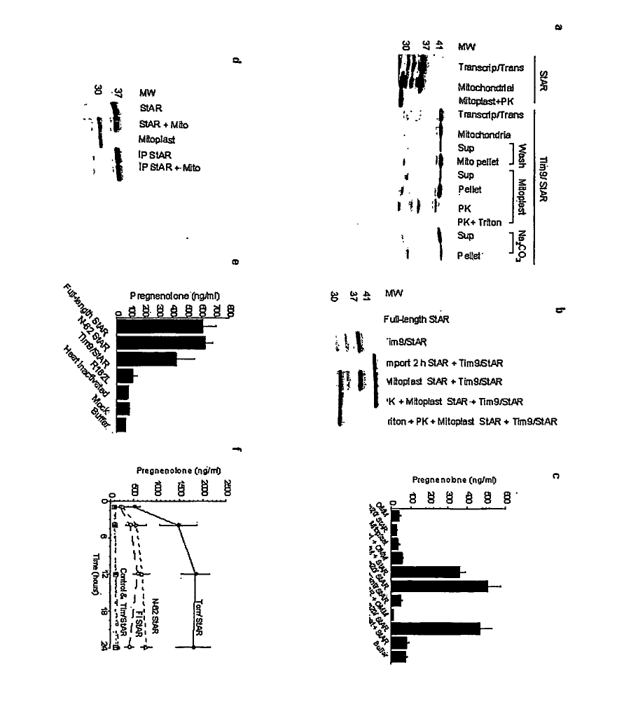

Mitochondrial import (as described above) followed by stripping away the OMM to produce mitoplasts showed that 37 kDa StAR was not retained but 30 kDa StAR was, validating the mitoplast preparation. Uncleaved Tim9/StAR (41 kDa) was associated with mitoplasts but was sensitive to proteinase K or could be released to the supematant by Na2CO3 showing it lies in the IMS (FIG. 2a). The presence of Tim9/StAR in the IMS did not disrupt the integrity of the OMM or mitochondrial function, as Tim9/StAR did not disrupt the import (FIG. 2b) or activity (not shown) of full-length StAR. When OMM and mitoplasts are separated, each component is steroidogenically inactive, irrespective of whether it contains a StAR construct. When mitochondria are reconstituted they are active with exogenously added StAR or when the OMM fraction contains Tom20/StAR; the presence or absence of Tim9/StAR in the mitoplast preparation contributes no activity and exogenously added StAR is inactive with mitoplasts in the absence of OMM (FIG. 2c). Although Tim 9/StAR was inactive in the IMS, it was active on the OMM. Tim9/StAR separated from reticulocyte lysate system by immunoprecipitation could not be imported (FIG. 2d) but elicited activity on isolated mitochondria. (FIG. 2e). Thus Tim9/StAR is inactive because of its IMS localization and is inherently inactive from misfolding.

Measurement of steroidogenic activity of isolated mitochondria following StAR importation shows the cell-free system faithfully recapitulates the key features of StAR biogenesis and activity observed in vivo (FIG. 2f). Mitochondria incubated with control cell-free transcription/translation system or that expressing Tim9/StAR yielded minimal steroidogenesis, but expression of either fill-length StAR or N-62 StAR increased steroidogenesis ˜6-fold, and expression of Tom/StAR increased activity 15-20 fold. Thus Tom/StAR is substantially more potent than full-length or N-62 StAR, both in cells (FIG. 1a) and in isolated mitochondria. Although some investigators categorize active and inactive forms of StAR on the basis of size (30 kDa vs. 37 kDa protein) our data show that StAR's activity is determined by its mitochondrial location rather than its size. This is consistent with recent data indicating that only newly synthesized StAR is active (Artemenko, I. P., et al., J Biol Chem 276, 46583-46596 (2001)). Thus FIG. 1 shows that StAR is active on the OMM and FIG. 2 shows it can not act in the IMS.

### EXAMPLE 3

### Association of StAR Activity with Mitochondrial Import

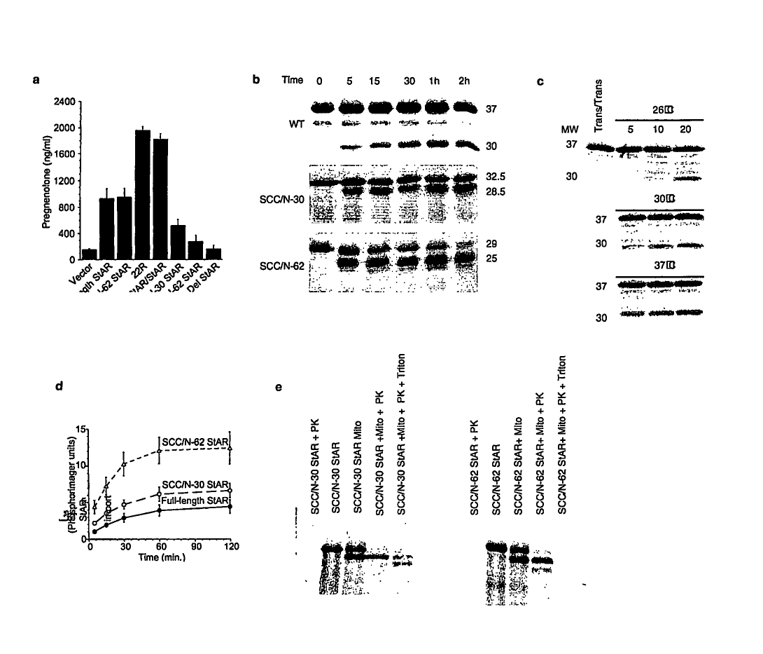

As StAR activity is confined to the OMM, slowing import should increase activity. StAR residues 63-188 are protease-resistant and may slow StAR's import (Bose, H. S., et al. Proc Natl Acad Sci USA 96, 7250-7255 (1999)), thus a second copy of 63-188 was added in front of 63-285 StAR (StAR/StAR). Conversely, to speed StAR's entry, StAR residues 1-30 (Scc/N-30 StAR) and residues 1-62 (Scc/N-62 StAR) were replaced with the 39AA leader of P450 scc, and residues 31-62 were deleted from wild-type StAR (del StAR). When transfected into COS-1 cells co-transfected with F2, StAR/StAR achieved activity equal to that with 22R-OH-cholesterol, Scc/N-30 StAR had about half the activity of full-length or N-62 StAR, and Scc/N-62 StAR and del StAR were equivalent to control (FIG. 3a). Thus constructs designed to slow mitochondrial entry increased activity and those designed to speed entry decreased activity. To estimate mitochondrial entry kinetics, we prepared each of these constructs by translation/transcription and incubated them with MA-10 cell mitochondria (FIG. 3b) at 26° C., which slows mitochondrial import, facilitating comparison between constructs (FIG. 3c). Phosphorimager quantitation of the uncleaved and the cleaved imported protein showed import speeds for Scc/N-62 StAR>>Scc/N-30 StAR>full-length StAR (FIG. 3d). Both the Scc/N-30 StAR and Scc/N-62 StAR constructs were imported into mitochondria, as evidenced by their cleavage from a full-length form to a protease-resistant intramitochondrial 30 kDa form (FIG. 3e). Thus StAR leader mutants that decreased activity speed mitochondrial import of the construct.

### EXAMPLE 4

### Full Length and N-62 StAR are Equally Active

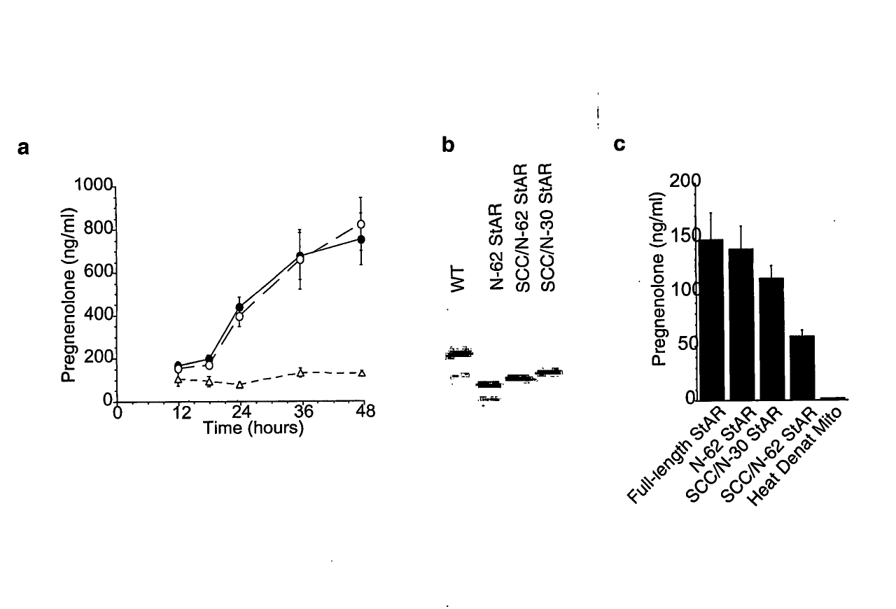

It has been suggested that the activity of N-62 StAR is an artifact due to overexpressession in transfected cells (Tsujishita, Y., Hurley, J. H., Nature Struct Biol 7, 408-414 (2000)). When COS-1 cells are transfected with equimolar amounts of expression vectors for full-length or N-62 StAR, the steroidogenic activity conferred to the cells is equivalent at all times measured after transfection (FIG. 4a). To ensure that this reflects the activity of equivalent amounts of protein, the amounts of wild-type, N-62, Scc/N-30 and Scc/N-62 StAR produced by in vitro transcription/translation were estimated by immunoblotting compared to known amounts of bacterially expressed N-62 StAR20 (FIG. 4b). When equal amounts of each protein were added to MA-10 cell mitochondria, full-length and N-62 StAR were equally active, Scc/N-30 StAR was slightly less so, and Scc/N-62 StAR had about half the activity of wild-type or N-62 StAR (FIG. 4c). Thus the activity of N-62 StAR expressed from transfected plasmids is equivalent to full-length StAR, and is not a pharmacological artifact.

### EXAMPLE 5

### Identification of an Outer Mitochondrial Membrane StAR Receptor Binding Protein

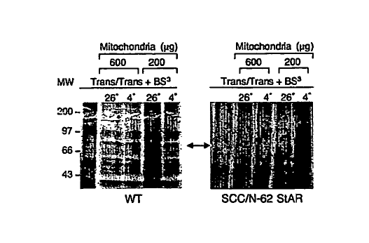

³⁵S labeled full-length StAR (WT) and SCC/N-62 StAR were incubated with a homobifunctional crosslinker, BS³, in presence and absence of isolated mitochondria obtained as described above at 26° and 4° C. A new protein cross-linked product of approximately 80 kDa is present only with the full-length StAR at both 26° and 4° C., but not in the absence of mitochondria (extreme left lane), or if the StAR signal sequence and amino acids 1-62 (containing the putative pause region) is replaced by that of side chain cleavage enzyme (SCC) (see FIG. 5, right hand panel). Thus this experiment indicates a likely OMM receptor for StAR that will be a useful target for drug development. The approximate molecular weight of this receptor is 45 kDa.

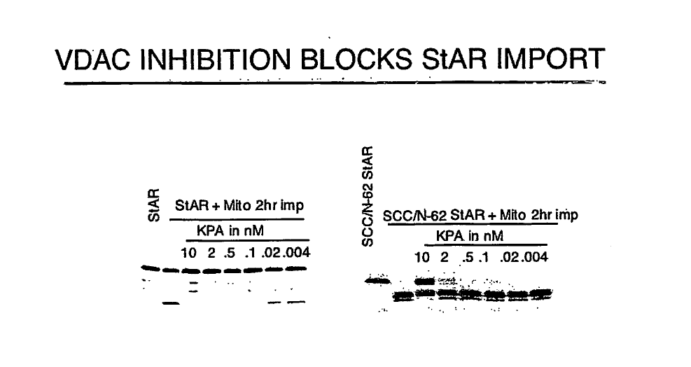

In order to confirm that binding of StAR to the receptor binding protein was StAR signal sequence-specific, inhibition of protein import into mitochondria was evaluated using König's polyanion, a VDAC-specific inhibitor. The polyanion is a copolymer of methacrylate, maleate and styrene in a 1:2:3 ratio and has an average molecular weight of 10,000. In addition to increasing voltage dependence as dextran sulfate does, the polyanion binds to and induces closure of VDAC even in the absence of a membrane potential (Colombini et al., (1987) Biochim. Biophys. Acta 905: 279-286). 50 μl of StAR or SCC-StAR translation product was mixed with 250 μg of mitochondria prepared by resuspending 10,000× g pellet from 10 million cell homogenate, import was carried out for 1 hr in the absence or presence of VDAC inhibitor Koenig's poly anion at 20 nM to 0.002 nM concentration points. After 1 hr at 26° C., the reaction was placed on ice and aliquots analyzed for import by signal cleavage and proteolysis as described above (see Methods). As shown in FIG. 6, import of StAR, but not of a chimera containing an SCC leader on a mature StAR sequence, was blocked by the VDAC-specific inhibitor indicating that VDAC represents an alternate mitochondrial import channel utilized by StAR and possibly other substrates. StAR import was 50% inhibited by less than 4 pM of König's polyanion. Fifty percent inhibition of SCC/N-62 StAR required 4 nM König's polyanion, more than a thousand-fold difference.

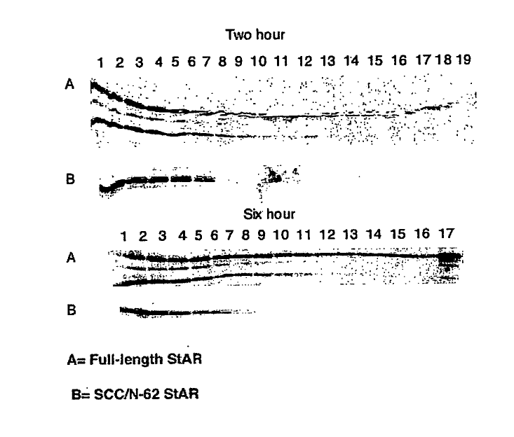

In order to further characterize the association between StAR and a receptor binding protein, migration of StAR and SCC-StAR on glycerol density gradients was evaluated. StAR and SCC-StAR translation products were imported into mitochondria as described above and aliquots of the import reaction analyzed on glycerol density gradients. Translation and import was carried out as described above and a 20 μL aliquot was taken, solubilized in 1% lauryl maltoside and applied to 10-30% glycerol gradients containing 750 mM amino caproic acid pH 7.4 detergent and centrifuged in a TLS55 rotor for 2 or 6 hours. 125 μl fractions were taken and analyzed by SDS PAGE. The results are shown in FIG. 7. StAR but not SCC-StAR displays a fraction of material associated with a high molecular weight protein complex that migrates within the gradient after 2 hrs and pellets after 6 hrs of centrifugation.

The proteins associated with the high molecular weight complex were identified using mass spectrometery. 100 μl translation product obtained as described above was solubilized with 1% maltoside and analyzed by native blue gel electrophoresis. Bands that coassociated with StAR but not SCC StAR were excised and analyzed by Matrix-assisted laser desorption ionization/Time of flight (MALDI/TOF). The following products were identified with StAR but not SCC StAR: VDAC1 and VDAC3, adenine nucleotide translocator, aldehyde dehydrogenase, ADP, ATP carrier protein and glucose regulatory protein 78 (GRP-78) indicating that the receptor binding protein for the StAR signal sequence is a complex that includes VDAC.

The above data establish: i) that StAR functions on or in the OMM, a location it maintains transiently; ii) that StAR's activity is proportional to its residency time on or in the OMM; iii) that the mitochondrial importation of StAR terminates its activity; iv) that sequences in the first 62 amino acids of StAR specifically slow StAR's mitochondrial import compared to the leader of P450scc and v) a likely OMM receptor for StAR. Because mitochondrial import is rapid, this unusual system permits rapid regulation of steroidogenesis in response to physiologic needs and establishes the rate of mitochondrial protein translocation as another site of physiologic regulation. These data indicate that StAR's activity is regulated by its rate of mitochondrial import. The identification of a receptor binding protein for the StAR leader sequence provides a useful target for drug development.

All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporate by reference.

The invention now having been fully described, it will be apparent to one of ordinary skill in the art that many changes and modifications can be made thereto without departing from the spirit or scope of the appended claims.

What is claimed is:

1. A method for identifying a cellular location that corresponds to the site of action of a cellular process, said method comprising:
expressing a key protein in said cellular process with a cell-free transcription/translation system comprising an organelle that is a putative site of action for said cellular process.

2. A method for identifying a mitochondrial compartment that corresponds to the site of biological activity of a mitochondrial protein, said method comprising:
cotransfecting a host cell with (a) a first nucleic acid encoding said mitochondrial protein and a means for immobilizing said mitochondrial protein in a mitochondrial compartment selected from the group consisting of the outer mitochondrial membrane, the inner mitochondrial membrane, the intramembranous space, and the matrix; and (b) a second nucleic acid encoding a reporter system for evaluating the biological activity of said mitochondrial protein whereby a first, second, third and fourth transfected host cell respectively are produced;
expressing said first and second nucleic acids whereby the amount of expression product of said reporter system produced by each transfected host cell as compared to a control transfected host cell expressing said second nucleic acid alone is indicative of the index of biological activity of said mitochondrial protein in each mitochondrial compartment.

3. The method according to claim 2, wherein said method further comprises:
measuring said biological activity in a cell-free system comprising mitochondria into which the expression products of said first, second, third and fourth transfected host cells have been imported.

4. A method for obtaining a receptor-ligand complex comprising a StAR mitochondrial leader sequence and a receptor for said leader sequence, said method comprising:
combining a functional StAR protein comprising said leader sequence and up to the full length of said StAR protein joined with a means of immobilizing said protein on the cytoplasmic side of the outer mitochondrial membrane with a source of said receptor; and recovering said receptor-ligand complex.

5. The method according to claim 4, wherein said receptor is contained in steroidogenic host cells.

6. The method according to claim 4, wherein said functional StAR protein is expressed in a cell free system.

7. An isolated mitochondrial StAR receptor binding protein comprising as a first subunit a voltage dependent anion channel (VDAC).

8. The isolated mitochondrial StAR receptor binding protein according to claim 7, wherein said VDAC comprises VDAC1 or VDAC3.

9. The isolated mitochondrial StAR receptor binding protein according to claim 7, wherein said binding protein comprises as one or more additional subunits one or more protein selected from the group consisting of an adenine nucleotide translocator, an aldehyde dehydrogenase, an ATP carrier protein and a glucose regulated protein 78.

10. The isolated mitochondrial StAR receptor binding protein according to claim 7, wherein said receptor binding protein is obtainable from a human steroidogenic cell.

11. An isolated complex comprising a StAR leader sequence and a StAR receptor binding protein according to any one of claims 7-10.

12. A recombinant non-steroidogenic host cell comprising:
a receptor for a StAR mitochondrial leader sequence.

13. A method for enhanced steroid production in a steroidogenic host cell, said method comprising:
transfecting said steroidogenic host cell with a nucleic acid encoding a biologically active StAR protein and a means for immobilizing said StAR protein on the cytoplasmic side of the outer mitochondrial membrane; and
growing said host cell whereby enhanced steroidogeneis is obtained.

14. The method according to claim 13, wherein said steroidogenic host cell is selected from the group consisting of Leydig cells, ovarian follicular cells, and endometrial cells.

15. A method for producing pregnenolone in a non-steroidogenic animal host cell, said method comprising:
cotransfecting said host cell with (a) a biologically active StAR protein or a second protein having a functional domain that is essentially identical with that of a StAR protein and has StAR-like steroidogenic activity and a means for immobilizing said StAR protein on the cytoplasmic side of the outer mitochondrial membrane; and (b) a second nucleic acid encoding a cholesterol side chain cleavage enzyme system;
growing said cells whereby pregenolone is produced.

16. The method according to claim 15, wherein said host cells are COS-1 cells.

17. The method according to claim 15, wherein said second protein is MLN64.

18. A method for screening for compounds that alter peripheral steroidogenesis, said method comprising:
contacting nonsteroidogenic cells comprising MLN64 that synthesize a steroid hormone or precursor thereof with a test compound, whereby a change in the rate or amount of synthesis of said hormone is indicative of a test compound that alters peripheral steroidgenesis.
